(19) United States
(12) Patent Application Publication
(10) Pub. No.: US 2017/0112634 A1
(43) Pub. Date: Apr. 27, 2017

(54) Objective, Real-Time Acoustic Measurement and Feedback for Proper Fit and Fill of Hip Implants

(71) Applicant: THE REGENTS OF THE UNIVERSITY OF CALIFORNIA, Oakland, CA (US)

(72) Inventors: Cameron Allan Gunn, Los Angeles, CA (US); George Wagdi Hafzalla, Huntington Beach, CA (US); Benjamin Christopher Bengs, Santa Monica, CA (US)

(21) Appl. No.: 15/315,619

(22) PCT Filed: Jun. 3, 2015
(86) PCT No.: PCT/US15/34064

§ 371 Date: Dec. 1, 2016

Related U.S. Application Data
(60) Provisional application No. 62/007,332, filed on Jun. 3, 2014.

(51) Int. Cl. A61F 2/46

(52) U.S. Cl. CPC A61F 2/4607; A61F 2/4657; A61F 2002/4671; A61F 2002/4681

(57) ABSTRACT

The present invention relates to a system and method for objective, real-time acoustic measurement and feedback for proper fit and fill of hip implants during total hip replacement surgery, or other bone implant surgical procedures.

### CROSS-REFERENCE TO RELATED APPLICATIONS

This application claims priority to U.S. Patent Application Ser. No. 62/007,332, filed Jun. 3, 2014, the entire contents of which are incorporated herein by reference.

### BACKGROUND OF THE INVENTION

Total hip replacement procedures seek to replace a hip joint that has deteriorated in its functionality, limiting a person's range of motion and weight bearing strength, in addition to causing significant pain. Total hip replacement typically involves removal of the femoral head, neck, and a portion of the top of the femur in order to replace these structures with prosthetic components.

Skeletal development and posture can be highly variable from person to person, while the prosthetic components are generally standardized. Thus, each hip replacement procedure is unique to the patient, and requires the surgeon to accommodate for these differences. A surgeon will typically measure both hip joints, including the neck-shaft angles, vertical offset, lateral offset, and leg length, prior to performing a total hip replacement procedure. These measurements allow the surgeon to match the replacement joint as closely as possible to the angles and dimensions of the original hip joint in order to achieve satisfactory range of motion, leg length, soft tissue tension, and stability.

During the procedure, the surgeon dislocates the joint, prepares the acetabulum with a cup prosthesis, and removes the femoral head and prepares the femur bone for receiving the prosthetic femoral stem, often by reaming and/or broaching the metaphyseal region and/or medullary canal to define a socket for receiving a prosthetic femoral stem. To set the prosthetic femoral stem, the surgeon must tamp the stem several inches inside the bone to ensure a firmly seated, proper fit.

While highly skilled, orthopedic surgeons currently are forced to rely solely on operative experience and manual assessment of the fit of implants in bone during an implant placement process, which inevitably leaves the procedure prone to technical error. These errors can lead to pain and sometimes require revision surgery that can greatly increase the patient's risk of death and also add to the cost burden of healthcare.

Although surgeons currently use X-ray templates to estimate the placement of implants prior to surgery, these 2-dimensional preparations fail to fully account for the 3-dimensional aspects of surgery as well as the surgeon's training background. Additionally, the use of cementless and porous implants to promote bone-implant integration relies primarily on the surgeon's ability to achieve an optimal fit of the implant for such integration.

Thus, there is a continuing need in the art for methods and devices for optimally fitting implants in bone. The present invention addresses this continuing need in the art.

### SUMMARY OF THE INVENTION

Described herein is a method for determining the position of an implant in bone. In one embodiment, the method includes: recording an auditory signal of one or more hammer hits during a hammering sequence of an implant into bone, measuring one or more acoustic features of the auditory signal, and classifying the one or more hammer hits based on the one or more acoustic features of the auditory signal, wherein the classification relates to the position of the implant in bone. Also described is a method for determining the fit of an instrument hammered into bone. The method includes the steps of collecting at least one audio signal indicative of a hammer striking an instrument into bone, segmenting the at least one audio signal into segments indicative of individual hammer strikes, classifying the segmented audio signals, and providing instruction to a user regarding the fit of the instrument within the bone. In one embodiment, the hammering sequence comprises the step of hammering a broach into cancellous bone. In one embodiment, the one or more hammer hits are classified according to the position of the broach in the bone. In one embodiment, the bone implant procedure is a hip arthroplasty. In one embodiment, the acoustic features correspond to compaction between a hammer and a broach introducer, compaction between a broach and cancellous bone, or compaction between a broach and cortical bone. In one embodiment, the method further comprises providing a feedback to a surgeon performing the bone implant procedure. In one embodiment, the feedback is an auditory signal corresponding to the position or fit of a broach in a bone. In one embodiment, the feedback is a visual signal corresponding to the position or fit of a broach in a bone. In one embodiment, the auditory signal recorded comprises at least three frequency bands. In one embodiment, the auditory signal recorded comprises frequency bands in ranges of about 1-2 kHz, about 2-4 kHz, and about 5-7 kHz.

A system for fitting an implant broach in a bone into bone is also described. The system includes, a microphone, a digital display, and a control unit including a microprocessor communicatively connected with the microphone and digital display, wherein when a broach is being hammered into a bone, the microphone is positioned to receive one or more auditory signals corresponding to a hammering sequence, and wherein the fit of the broach in the bone is determined by analysis of the one or more auditory signals by the microprocessor and reported via the digital display. In one embodiment, the microphone is suitable for receiving the one or more audio signals from a position outside of the sterile zone in an operating room. In another embodiment, the microphone is contained in a portable housing. In another embodiment, the digital display provides an auditory feedback. In another embodiment, the digital display provides a visual feedback. In another embodiment, the control unit is connected wirelessly with the microphone. In another embodiment, the one or more auditory signals correspond to compaction between the hammer and a broach introducer, compaction between the broach and cancellous bone, or compaction between the broach and cortical bone. In another embodiment, the one or more auditory signals analyzed comprises at least three frequency bands. In another embodiment, the one or more auditory signals analyzed comprises frequency bands in ranges of about 1-2 kHz, about 2-4 kHz, and about 5-7 kHz.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic representation of source separation, wherein a loose fit (A) of broach and bone generates a different vibration than a snug fit (B) of broach and bone.

FIG. 2 is a schematic of an exemplary system of the present invention.

FIG. 3 is a flowchart outlining an exemplary process performed by the system of the present invention.

FIG. 4, comprising FIGS. 4A-4C, is a set of graphs depicting the process of hit detection. FIG. 4A is a plot that shows the magnitude of a raw section of audio, FIG. 4B shows the envelope from the total variation smoothing method, and FIG. 4C shows a “1” at the time that a hit is detected by the deadband threshold.

FIG. 5, comprising FIGS. 5A and 5B, are exemplary reporting visualizations to a user of the system. FIG. 5A is a visualization of the cepstral coefficients, which FIG. 5B is an exemplary visualization of cepstral coefficients and vibrating mechanical systems.

FIG. 6 is a flowchart of an exemplary method of the present invention.

FIG. 7, comprising FIGS. 7A-7C, depicts data demonstrating the percent of energy of a broach hit in the 1-2 kHz band versus the number of occurrences.

FIG. 7A is a graph depicting energy from size 2-5 broach hits (last 10 hits). FIG. 7B is a graph depicting energy from size 6 broach hits (last 10 hits). FIG. 7C is a graph depicting energy from size 7 broach hits (last 10 hits).

FIG. 8 is a graph depicting data demonstrating resonance spectra for the final two hits in a hammering sequence.

FIG. 9 is a graph depicting the SVM separation of size 7 broach hits.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical orthopedic implant surgery and acoustic measurements. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The terms “patient,” “subject,” “individual,” and the like are used interchangeably herein, and refer to any animal amenable to the systems, devices, and methods described herein. Preferably, the patient, subject or individual is a mammal, and more preferably, a human.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### DESCRIPTION

The present invention relates to a system and method for improving the fit of a bone implant in a subject. As contemplated herein, the system and method utilizes acoustic analysis to improve the fit of a femoral stem inside the femoral cavity during hip arthroplasty, or any other procedure requiring fitting an implant in a subject.

In one embodiment, the system and method relies on providing a physician with objective feedback during joint replacement surgeries so that surgeons can optimally insert implants. The system and method can result in a decreased number of revision surgeries and the risk of mortality due to improperly implanted prosthesis, thereby reducing healthcare costs and potentially saving patient lives.

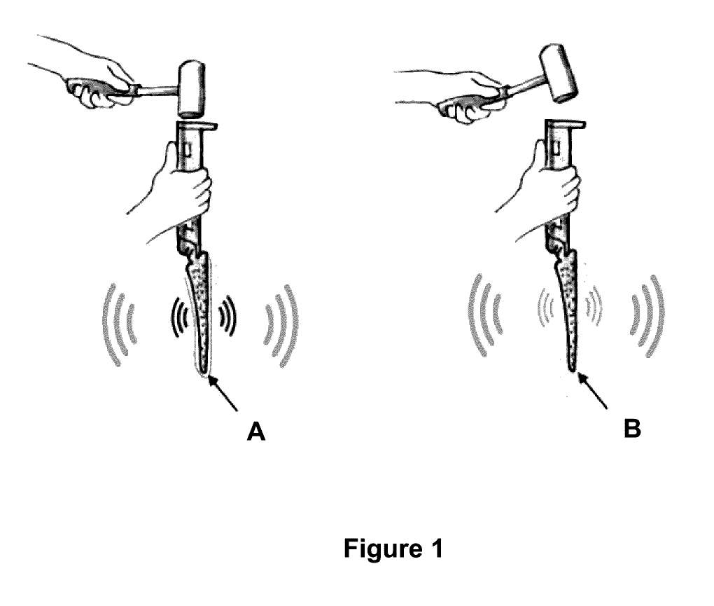

The present invention helps to improve implant fit by providing feedback to a surgeon during a broaching procedure by analyzing and classifying acoustic features generated from hammering the broach into cancellous bone. For example, as shown in FIG. 1, the underlying mechanism is blind source separation/detection. In a suboptimal, i.e. loose fit (A), the broach and bone are not well aligned and hence vibrate as two distinct mechanical systems. In an optimal, i.e. snug fit (B), on the other hand, broach and bone are well aligned and vibrate as a single dynamical system. By detecting the presence (or absence) of this separation, a recommendation on the quality of fit may be provided.

The systems and methods presented herein offer several advantages, including, but not limited to: 1) optimized fit and fill of a femoral stem inside the femoral cavity, corresponding to the position which will be taken up by the actual implant stem so that the broach provides a true gauge of the position of the implant femoral stem after implant; 2) acoustic analysis is a non-invasive aid that supplements the surgeon's sensory information to ensure the best surgical outcome; 3) provides an auditory feedback to the surgeon so that the surgeon can indirectly confirm when the broach arrives at the appropriate position within the femoral cavity, where the broach is in maximum contact with compact bone; 4) can be used within current surgical workflows and procedures to offer significant feedback information while minimizing extra surgical time; 5) can be used as a surgical training tool for new surgeons or students; 6) can be integrated with other modalities of intraoperative information collected to enhance the 3-dimensional aspects of surgery.

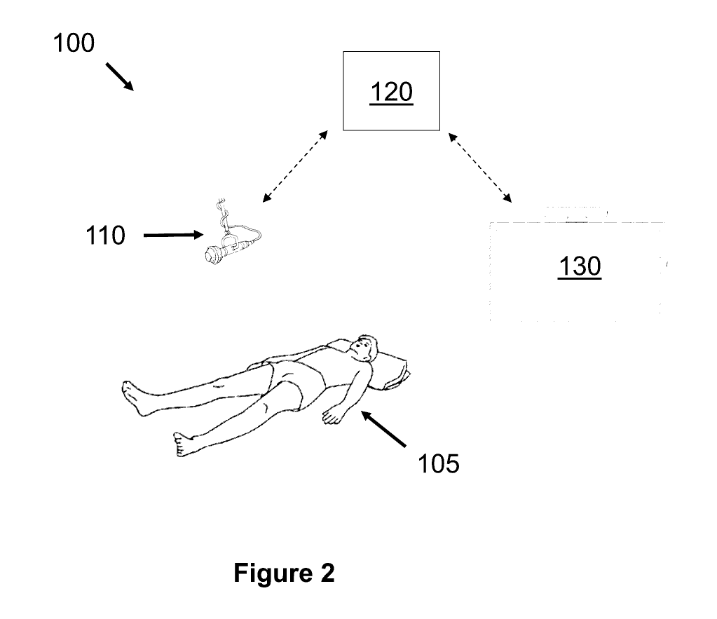

In one embodiment, an exemplary system 100 is illustrated in FIG. 2. System 100 may generally include one or more high-fidelity microphones 110, one or more digital displays 120, and a control unit 130. Depending on the type of components used, it should be appreciated that all or any portions of microphone 110, display 120 and control unit may be integrated or merged together into multi-functional devices, as would be understood by those skilled in the art. The system and method described herein can be used with any currently available broach and associated tools known in the orthopedic device industry. It should be appreciated that various details and designs of system 100 can be modified for use in orthopedic procedures other than hip arthroplasty. The spirit and scope of the invention, as set forth herein, is constructed to be a platform technology that is expandable to other procedures. For instance, the scope of the method can be modified for use in shoulder, elbow, wrist, knee and ankle surgeries or partial hip replacement, without limitation.

Microphone 110 can be placed inside or outside of a sterile zone in the operating room to capture real-time sound information produced between compaction of hammer and broach introducer, broach and cancellous bone, and between broach and cortical bone of a subject 105. There is no limitation to the number and type of microphones used, and accordingly any standard and preferably any high-fidelity microphone may be used as would be understood by those skilled in the art.

Digital display 120 may be any computing or thin client device capable of receiving and displaying audio and/or visual data and images relating to the sounds of the surgical procedure being performed. Without limitation, digital display 120 may be a monitor, a television, a smartphone, a tablet or any other digital display screen capable of presenting both audio and visual data. In other embodiments, digital display 120 may include multiple displays that show the same or different images received from control unit 130. In still other embodiments, digital display 120 may be a computing device capable of receiving signals directly from microphone 110 components.

A control unit 130 may include one or more computing devices which run the software and algorithms described elsewhere herein. Exemplary computing devices include, but are not limited to, a computer, desktop, laptop, tablet, phone, and the like. The computing devices may include at least one processor, standard input and output devices, as well as all hardware and software typically found on computing devices for storing data and running programs, and for sending and receiving data over a network. Control unit 130 may be connected to microphone 110 components and digital display 120 via a communications network, and thus receives the audio signal from microphone 110, processes data and transmits to digital display 120 for visualization and presentation of acoustic feedback to form an open or closed loop feedback system. For example, after receiving the audio signal from microphone 110, the audio signal may be recorded and analyzed to extract acoustic features that are used in a predictive model for monitoring the broaching procedure. An algorithm resident on or run by control unit 130 can be used to analyze and/or classify the acoustic features of the audio signal. Processed data may then be sent to digital display 120 for presenting or otherwise reporting the output of the processed information.

Thus, in one embodiment, the high-fidelity microphone may be positioned above a patient during total hip replacement surgery. After the head of the femur has been removed from the patient, the surgeon hollows out the femoral cavity by hammering in increasingly larger broaches, which are the same size and shape as their corresponding implant. Audio from these hammering sequences may be recorded by the microphone and streamed to the control unit in real time. Recording may also occur at the control unit directly. There, audio may be segmented into individual hammer hits, processed into acoustic features of interest, and then classified as a good or poor fit by a machine learning technique, optionally trained on predetermined patient data. Decisions may be weighted and combined, and the acoustic estimate of fit and fill may be relayed to the operating surgeon by the digital display and auditory feedback.

The software running on or by system 100 may include a software framework or architecture that optimizes ease of use of at least one existing software platform, and that may also extend the capabilities of at least one existing software platform. The software provides applications accessible to one or more users (e.g. patient, clinician, etc.) to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system. The GUI may display, for example, information regarding the calculated acoustic features determining the fit and stability of the broach in the femur during surgery.

Presentation of data through the software may be in any sort and number of selectable formats. For example, a multi-layer format may be used, wherein additional information is available by viewing successively lower layers of presented information. Such layers may be made available by the use of drop down menus, tabbed pseudo manila folder files, or other layering techniques understood by those skilled in the art.

The software may also include standard reporting mechanisms, such as generating a printable results report, or an electronic results report that can be transmitted to any communicatively connected computing device, such as a generated icon, text, email message or file attachment. Likewise, particular results of the aforementioned system can trigger an alert signal, such as the generation of an alert icon, email, text or phone call, to alert a patient, doctor, nurse, emergency medical technicians, or other health care provider of the particular results.

In certain embodiments, data is transferred from microphone 110, control unit 130, and digital display 120 using either wired or wireless communication. Wireless communication for information transfer to and from system 100 components may be via a wide area network and may form part of any suitable networked system understood by those having ordinary skill in the art for communication of data to additional computing devices, such as, for example, an open, wide area network (e.g., the internet), an electronic network, an optical network, a wireless network, a physically secure network or virtual private network, and any combinations thereof. Such an expanded network may also include any intermediate nodes, such as gateways, routers, bridges, internet service provider networks, public-switched telephone networks, proxy servers, firewalls, and the like, such that the network may be suitable for the transmission of information items and other data throughout system 100. For example, data transfer can be made via any wireless communication and may include any wireless based technology, including, but not limited to radio signals, near field communication systems, hypersonic signal, infrared systems, cellular signals, GSM, and the like. In some embodiments, data transfer is conducted without the use of a specific network. Rather, in certain embodiments, data is directly transferred to and from the system 100 components.

In one embodiment, system 100 includes software designed to collect audio data from hammering sequences and segment the data into individual hammer hits, process the data into acoustic features of interest, and then classify the data as a good or poor fit by a machine learning algorithm optionally trained on patient data. The software is capable of making decisions that are weighted and combined, and the acoustic estimate of fit and fill may be relayed to the operating surgeon.

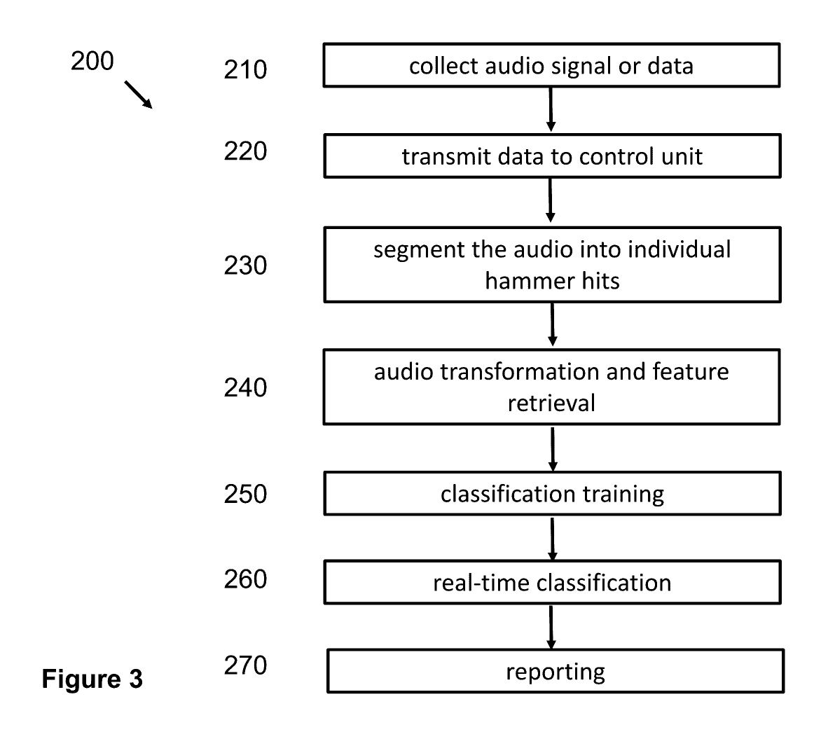

As shown in FIG. 3, the system may perform the following procedural steps of process 200. First, at step 210, the system collects audio signal or data. In one embodiment, audio from mallet hits on a broach during surgery are collected from the microphone in the operating room. For example, the microphone may be affixed to an operating room surgical light over the patient's hip, but above the sterile field for optimal sound recording. Additionally, the microphone gain may be optimally tuned to receive sound with audio data that can be differentiated between relevant signals and background noise. In one embodiment, the audio collected is received continuously from the microphone. At step 220, the audio recording is streamed or otherwise transferred to the control unit for further processing via a wireless transfer protocol, such as Bluetooth. In other embodiments, alternative approaches to sound recording and transfer include a wired microphone recording using a cable, or a microphone attached to a device or wearable mobile device. Sound recordings can also be recorded and then transferred to the control unit at a later time rather than in real time. Alternate fixtures in the operating room can be used to hold the microphone above the patient. These can be ad hoc and room-specific, or a custom-designed fixture to the ceiling designed specifically for the microphone.

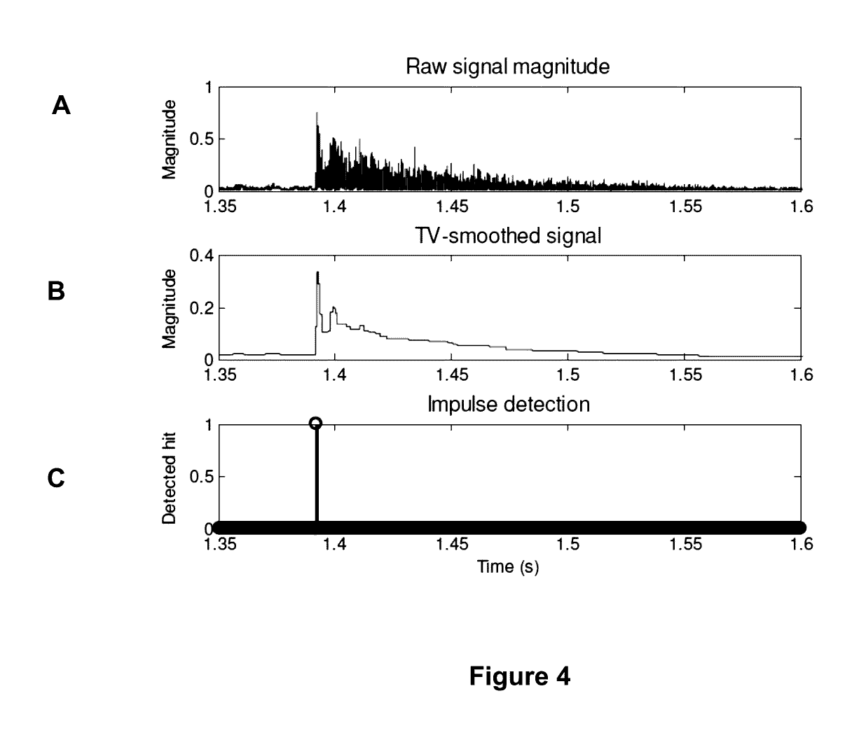

At step 230, the audio is segmented into individual hammer hits. Generally, as shown in FIG. 4, the process of hit detection is depicted, illustrating the magnitude of a raw section of audio (4A), the envelope from the total variation smoothing methods (4B), and the deadband threshold (4C). For example, first, the audio is separated into sequences of hits. These sequences correspond to the surgeon hammering the broach into the femur, and then removing it repeatedly engaging a release latch. There can be several sequences per broach size and varies by surgeon. For example, sequence segmentation may be performed by identifying a region of four seconds in which the audio exceeds 50% of the normalized amplitude limit that the microphone is tuned to. Each of the individual hammer hits may be individually separated in real time, as follows: If the sequence signal is stereo, it is mono mixed. The signal is decimated by preserving only the largest absolute magnitude point out of each 100 points (i.e. decimated by a factor of 100). The envelope of the signal is calculated using total variation enveloping. This method smooths the decay component of the signal, while preserving the sharp transition at the moment of impact. The following optimization problem is solved using any well-established convex optimization method, such as gradient descent, Newton's method, or interior point methods:

$$minimize {‖ x - \hat{x} ‖}_{2} + y {\sum }_{i = 1}^{n - 1} \vert {x}_{i} - {x}_{i + 1} \vert$$

where x is the known raw signal, \hat{x} is the desired smooth signal variable being solved, and γ is a smoothing constant currently set to 2.5×10⁻². The signal used to detect transition between hammer hits is \hat{x}.

A deadband is defined between 15% (lower band) and 25% (upper band) of the normalized amplitude limit of the signal. There is judged to be a transition to a new hammer hit if the envelope signal falls below the lower band and then subsequently rises above the upper band. Signals are defined as the raw (unsmoothed) audio between two transitions, and are individually stored.

In another embodiment, sequence segmentation can be alternatively approached by identifying key acoustic features of the release latch. These segments are easily distinguished in the time-domain signal. The discreet Fourier transform (DFT) of each identified impulse can be taken, and key spectral features can be used to classify whether an impulse is a hammer hit, or a release latch. A support vector machine (SVM), kernel machine or similarly simple machine learning technique can be trained to classify this difference, and can take a moving average of the last several hits to decide when the broaching finished, and when the releasing began.

Individual hit segmentation can also be approached by analyzing the signal energy in a given time window. As the signal decays, power should decrease, ensuring that the first half of the signal contains more power than the second half. Segmentation could be longer or shorter than four seconds. The sequences can be analyzed in stereo mix instead of mono. Deadband limits and smoothing constant values can be tuned when additional data is gathered. Other smoothing functions can be used to find the envelope of the curve, such as quadratic smoothing. The optimization problem above can also be solved by computing the dual of the problem.

At step 240, the audio is transformed and selected features are retrieved. In one embodiment, the DFT is taken using any well-established fast Fourier transform (FFT) algorithms such as Cooley-Tukey, Bluestein, and Rader's FFT. Audio features that contain fit information are extracted. The signal power in the 1-2 kHz, 2-4 kHz, and 5-7 kHz frequency bands are recorded. Every soundbite of a hammer hit is hence transformed into a 3×1 vector. Power is calculated as the square of the integral of (area under) the DFT in the given frequency band. In another embodiment, additional acoustic features for classification include power in lower bands; decay rates of both the signal and of particular harmonic regions; zero-crossing rate; and cepstral analysis. Time domain analysis and wavelet transform analysis may also be conducted. For example, mel frequency cepstral coefficients (MFCCs) may also be used as a measure of fit. The processing to obtain the MFCCs may include: pre-emphasis using a first-order high-pass filter to accentuate high-frequency components of the signal; taking the short time fourier transform of a windowed signal; transforming the frequency scale to the Mel scale where spectrum power is computed over multiple bands; taking the logarithm of powers in each band; and taking the discrete-cosine transform for de-correlation and compression. It should further be appreciated that variations of the above example are likewise feasible, without limitation, including alternative parameter choices, transforms, frequency scales and the like.

At step 250, the system may optionally perform classification training. In one embodiment, the techniques described herein are performed on a separate machine prior to implementing the system onto its final platform. Hence, these calculations are not performed in real-time during surgery.

1-2 kHz, 2-4 kHz, and 5-7 kHz band powers are treated as points in 3-dimensional space. Machine learning is employed for a supervised learning problem. An optimal separating plane is constructed between data known to be prior to final fit and data known to be the final fit, and shown by x-ray to be suitable fit, all data being from the first five hits of each sequence. The separating plane is constructed using a soft-threshold support vector machine. The following problem is optimized:

$$minimize μ ( {a}^{T} {a}_{i} ) + {\sum }_{i = 1}^{n} {u}_{i} subject to {s}_{i} ( {a}^{T} {x}_{i} - b ) \leq {u}_{i} - 1 , \\ i ∈ \{ 1 , 2 , 3 … n \}$$

where xᵢ is the known size vector representing point i of n in 3-dimensional space; a and b form the plane of optimal separation a^{T}x=b that is the desired solution to the problem sᵢε{−1,1} is a known binary label given to data point i (−1 if it is poor fit data, and 1 if it is optimal fit data); uᵢ is the penalty applied to point i if it falls on the wrong side of the separating plane, which is solved as part of the problem; and μ is the penalty constant, controlling the tradeoff between margin separation and misclassification of points, currently at a value of 0. This convex optimization problem is also solved using any of the well-established methods such as interior point, Newton's method, or gradient descent. The outcome of this problem is the plane defined by a and b and expressed as a^{T}x=b.

In another embodiment, the SVM can be solved for any arbitrary list of features, creating a hyperplane. Redundant features can be identified and removed using dimension reduction techniques such as principal component analysis and linear discriminant analysis. The plane of best separation can be solved by computing the solution to the dual of the above optimization problem.

A large number of machine learning techniques can be used to solve this problem. These include parametric methods and maximum likelihood estimation; hidden Markov models; kernel machines; k-nearest neighbors algorithms; and artificial neural networks. If parametric methods are used, prior distributions can be used to provide patient specificity. This prior data could come from earlier sizes of broaches, or from earlier broaching sequences with the same size of broach. The number of initial hits used to make a decision can be tuned. Alternatively, all hits or final hits can be used as classifying information.

When a more specific classification is desired, all of the features described herein may be considered. The dimension of the features may be reduced by using a dimensionality reduction technique, such as principal component analysis (PCA) or linear discriminant analysis (LDA), and subsequently passed to a classifying algorithm. As such, discriminating features may be identified automatically. For example, a given frequency band may offer information about quality of fit for given for subjects that are of young age, male and overweight, while a different frequency band may be discriminating for subjects that are of older age, female and average weight. Again, an SVM may be used as a classifying algorithm. In other embodiments, alternative implementations include kernel based extensions of an SVM (such as a Gaussian kernel or a polynomial kernel), linear discriminant analysis and kernel discriminant analysis, and K-nearest-neighbor classification.

Once the classifier has been trained, the projection matrix for dimensionality reduction along with the separating surface may be used to classify the quality of hits of new, compatible, patients. Compatibility, as used herein, may refer to similarity in terms of gender, age, weight, etc. Along with a decision (poor or good fit), the classifier may return a confidence measure (in terms of distance from the separating surface). This may be reported to the surgeon as a more concise quality of fit metric. It is furthermore aggregated over time (average or median) to provide a smoother and more confident classification. It should be appreciated that just one, multiple or all possible features may be included. In such cases where numerous features are included, the algorithms described herein may weigh features according to their importance, or remove one or more features altogether. Accordingly, at step 260, the system performs real-time classification. In one embodiment, as described in previous sections, audio is collected and segmented into audio sound bites of individual hammer hits. These hammer hits are transformed into a feature vector that can be treated as a point; a plane of optimal separation exists in relation to these points. The first five hammer hits in a sequence are placed in the 3D feature space and compared to the separating plane. Points indicating good fit are assigned an outcome scalar 1, and for poor fit, −1. The final outcome is calculated as the median outcome. In another embodiment, measures of distance between each point and the separating line can be constructed using well-established vector geometry. Examples of these measures are norms, such as the 1-norm, 2-norm (Euclidean distance), and co-norm. These distances can be used as a level of decision confidence. Using decision confidence, rather than taking the median of outcomes, the final outcome can be the confidence-weighted median of each outcome scalar. As with the training data, the number of initial hits used to make a decision can be tuned. All hits or final hits can be used as data points for classification.

At step 270, the results are reported to the surgeon. In one embodiment, the user interface can be presented on the digital display. 2-dimensional data of the 1-2 kHz and 5-7 kHz components can be plotted alongside the line of best separation in these two dimensions, in real-time to allow surgeons to make a more informed clinical judgement during surgery. The display may also have a dynamic sliding scale that can indicate whether: 1) the surgeon needs to continue broaching, 2) the surgeon has achieved optimal cortical loading, or 3) that the surgeon is at risk of over-broaching and may cause a fracture in the bone. The interface can also have options for surgeons to specify the hip replacement brand, type of broach being used, and the surgeon name for the purposes of filing patient medical records.

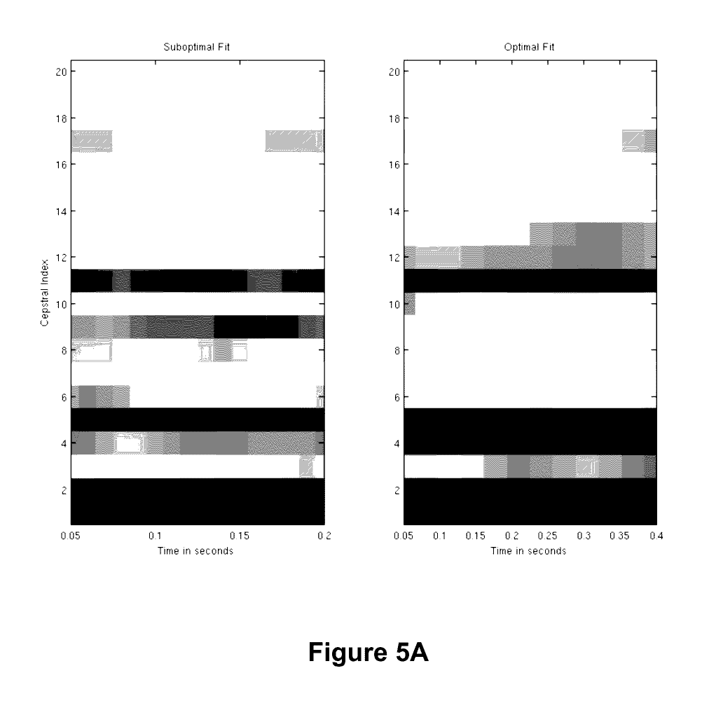

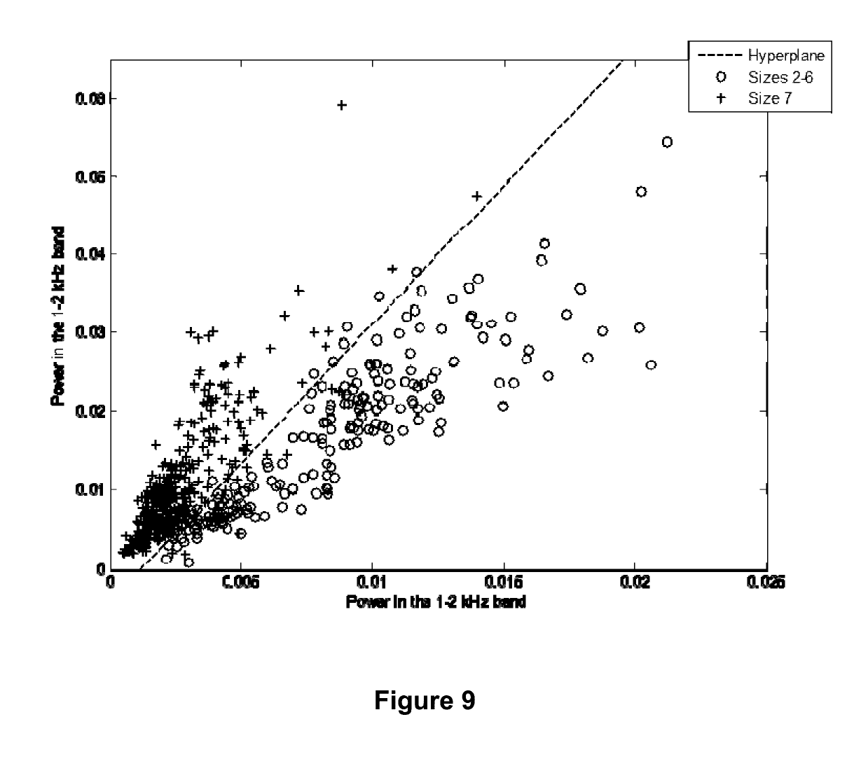

As contemplated herein, the surgeon or other user may receive auditory and/or visual feedback in real time. Accordingly, multiple displays or views can be implemented in the software described herein for the user to proper assess analysis for all available features. In one embodiment, the surgeon may be presented with a visual representation of some or all of aforementioned features or alternatively a subset or lower dimensional projection thereof. This aids in the determination of quality of fit. For example, the system may present the surgeon with a plot of cepstral coefficients averaged over the last series of hits for smoothing, such as in FIG. 5A, or the classification by filter bank as shown in FIG. 9. In another example, the display may additionally track the progress of the vibrating mechanical system(s), as shown in FIG. 5B. Such presentation may be beneficial because the system is capable of recognizing that the broach and bone vibrate as a single mechanical system, meaning that cortical interfacing has been reached.

In another embodiment, the user interface can extend beyond the 3 stages listed in the previous section (i.e., continue broaching, optimal loading achieved, etc.) and vary in detail. Other options for the user interface include an auditory feedback signal that would notify the surgeon on the surgery's progress. For example, the auditory signal can be ongoing or be a brief alert at each of the 3 stages as listed in the section above. The user interface can also include additional data, such as the spectral graph or data as a 2-D picture of key acoustic features, or the display should have options to access such additional data.

Other non-mutually exclusive ways to present the data on the interface can rely on color coding, displaying an anatomical animation of the fit and fill of broach to bone and the confidence interval, or providing a scale in which the larger the scale the more confident the data shows good fit. Additionally, the visual display can be a mobile device such as a smart phone or tablet, a surgical computer monitor, Google Glass or other wearable device, or any other similar device.

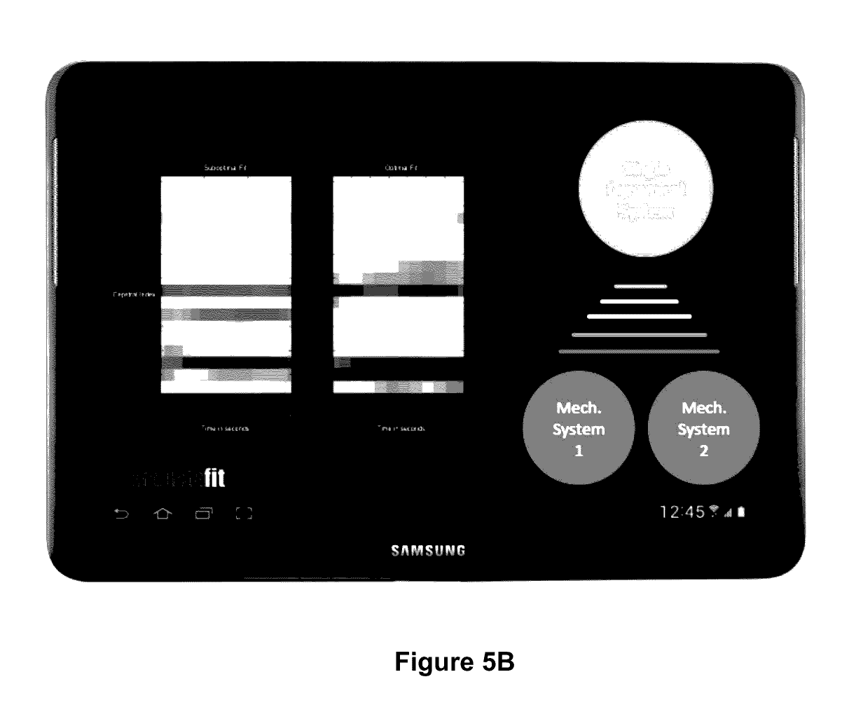

Accordingly, the system and processes described above can be further described in the following exemplary method, presented as method 300 in FIG. 6. First, the step of recording an auditory signal of one or more hammer hits during a hammering sequence of an implant into bone is performed 310. Next, the step of measuring one or more acoustic features of the auditory signal is performed 320. Subsequently, the step of classifying the one or more hammer hits based on the one or more acoustic features of the auditory signal is performed 330. Lastly, the step of correlating the classification to the position of the implant in bone is performed 340.

For example, in another embodiment of the method of the present invention, an auditory signal associated with the impact between the femoral component of a hip prosthesis and the patient's femur is measured. The impact data is acquired and analyzed using a suitable device. Metrics based on acoustic features are calculated to determine the fit and stability of the broach in the femur. Results are classified and outputted to a user interface to assist the surgeon. These results can then be combined with visual information in an open/closed loop to provide feedback to the surgeon.

In yet another embodiment, the detection and classification information may be relayed to external software, such as a computer-assisted surgical system. The software application for hit detection and classification may thus provide an application program interface (API) through which external software can query and retrieve information. This API may be made available through conventional programming languages, including but not limited to C, C++, Java, and Python. The API may also contain routines to request any information from the classification software, which is returned, either discretely per request, or streamed continuously, to the external software package. The external software package can be an electronic health record (EHR) program, a surgical robotic system, or any other embodiment of a computer-assisted surgical system as would be understood by those skilled in the art.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

A prototype was constructed comprising a microphone and computing system with display, running software and algorithms resulting in the following data:

### Audio Processing

Audio data was separated into individual hammer hits, which were analyzed separately. Separation was performed by downsampling the signal by a factor of 100 samples per sample, smoothing an incoming signal using total variation reconstruction, and using a deadband threshold to detect the impact of the hammer in the audio.

### Feature Extraction

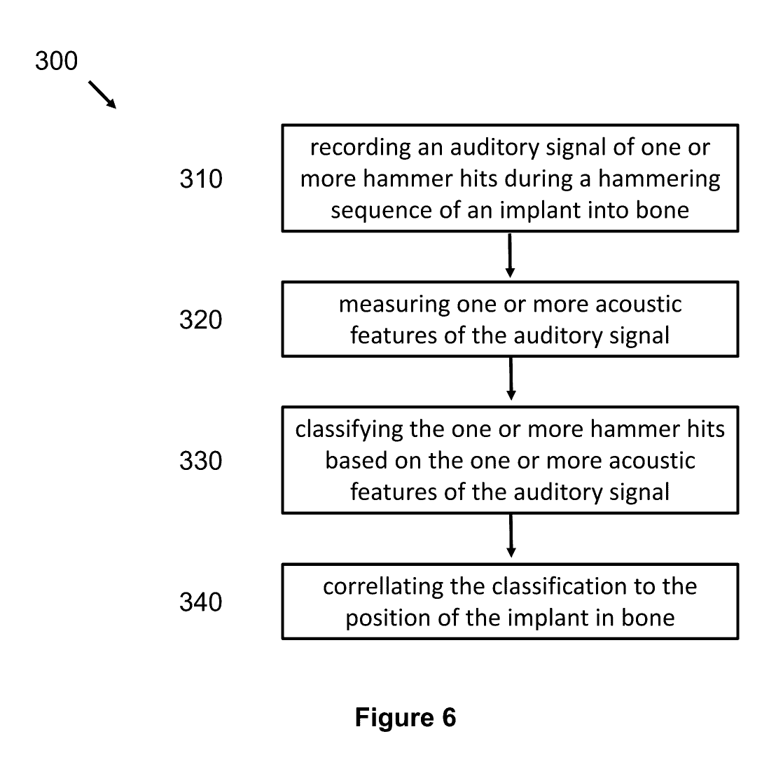

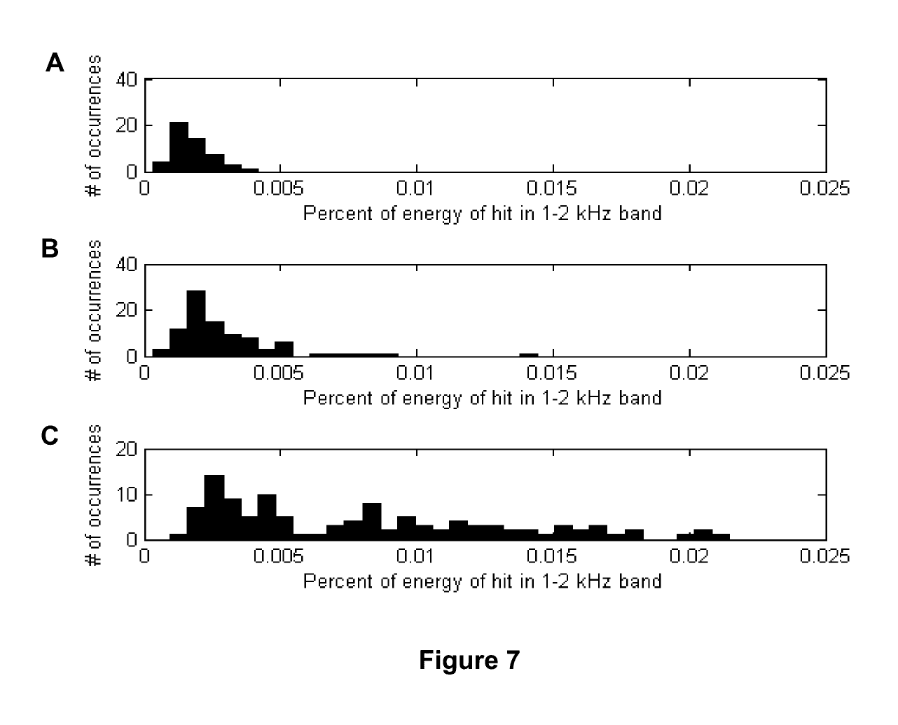

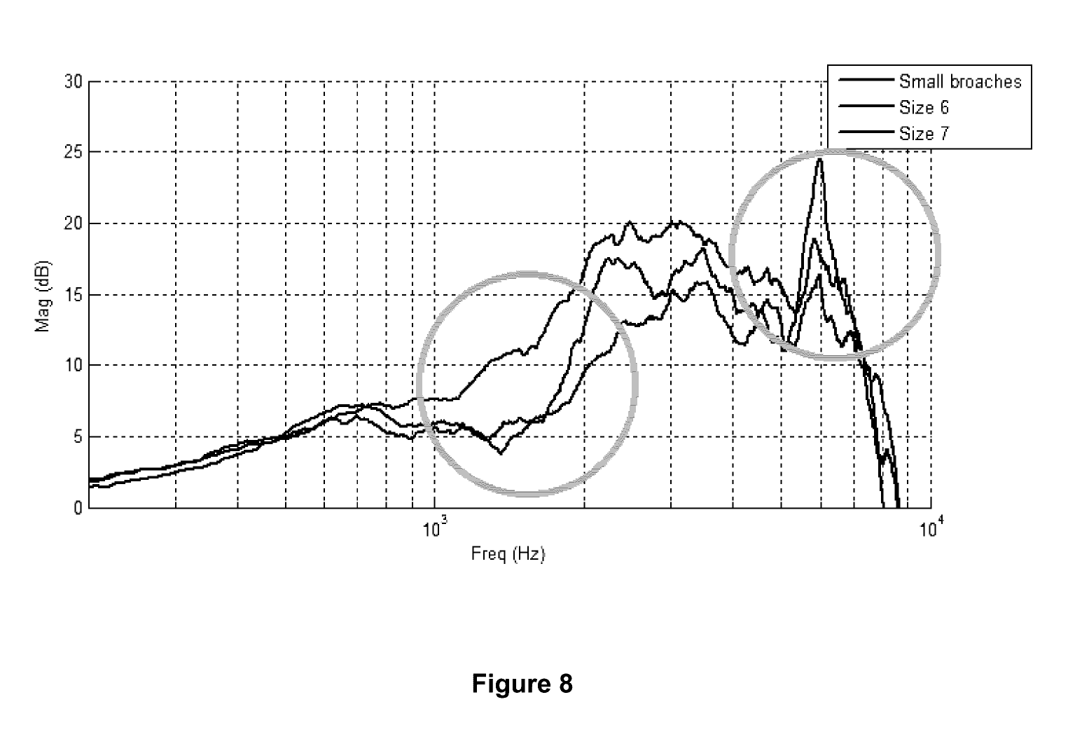

Audio was transformed by applying a Hanning window to the signal, and then taking the discrete Fourier transform (DFT). Signal power in frequency bands of interest, time-domain decay of these components, and other features such as entropy were extracted for use as classifying features (FIG. 7). The signal power in the 1-2 kHz and 5-7 kHz bands in particular were indicative of whether a broach was fully seated in the femur. (FIG. 8)

### Classification

Machine learning algorithms were used to classify signals based on the above features, providing robustness against variations in environmental noises, patient conditions, and broach brands. Specifically, support vector machines (SVMs), k-nearest neighbors, and artificial neural networks were used to classify the metrics into distinct categories (FIG. 9). The algorithm was trained during the design process, and a resulting classifying algorithm was integrated into a software package. The algorithm analyzed preliminary patient data to account for variations due to patient specificity.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for determining the position of an implant in bone, comprising:
recording an auditory signal of one or more hammer hits during a hammering sequence of an implant into bone,
measuring one or more acoustic features of the auditory signal, and
classifying the one or more hammer hits based on the one or more acoustic features of the auditory signal, wherein the classification relates to the position of the implant in bone.

2. The method of claim 1, wherein the hammering sequence comprises the step of hammering a broach into cancellous bone.

3. The method of claim 2, where the one or more hammer hits are classified according to the position of the broach in the bone.

4. The method of claim 1, wherein the bone implant procedure is a hip arthroplasty.

5. The method of claim 1, wherein the acoustic features correspond to compaction between a hammer and a broach introducer, compaction between a broach and cancellous bone, or compaction between a broach and cortical bone.

6. The method of claim 1, further comprising providing a feedback to a surgeon performing the bone implant procedure.

7. The method of claim 6, wherein the feedback is an auditory signal corresponding to the position or fit of a broach in a bone.

8. The method of claim 6, wherein the feedback is a visual signal corresponding to the position or fit of a broach in a bone.

9. The method of claim 1, wherein the auditory signal recorded comprises at least three frequency bands.

10. The method of claim 1, wherein the auditory signal recorded comprises frequency bands in ranges of about 1-2 kHz, about 2-4 kHz, and about 5-7 kHz.

11. A system for fitting an implant broach in a bone, comprising:
a microphone,
a digital display, and
a control unit including a microprocessor communicatively connected with the microphone and digital display,
wherein, when a broach is being hammered into a bone, the microphone is positioned to receive one or more auditory signals corresponding to a hammering sequence, and wherein the fit of the broach in the bone is determined by analysis of the one or more auditory signals by the microprocessor and reported via the digital display.

12. The system of claim 11, wherein the microphone is suitable for receiving the one or more audio signals from a position outside of the sterile zone in an operating room.

13. The system of claim 11, wherein the microphone is contained in a portable housing.

14. The system of claim 11, wherein the digital display provides an auditory feedback.

15. The system of claim 11, wherein the digital display provides a visual feedback.

16. The system of claim 11, wherein the control unit is connected wirelessly with the microphone.

17. The system of claim 11, wherein the one or more auditory signals correspond to compaction between the hammer and a broach introducer, compaction between the broach and cancellous bone, or compaction between the broach and cortical bone.

18. The system of claim 11, wherein the one or more auditory signals analyzed comprises at least three frequency bands.

19. The system of claim 11, wherein the one or more auditory signals analyzed comprises frequency bands in ranges of about 1-2 kHz, about 2-4 kHz, and about 5-7 kHz.

20. A method for determining the fit of an instrument hammered into bone, comprising:
collecting at least one audio signal indicative of a hammer striking an instrument into bone;
segmenting the at least one audio signal into segments indicative of individual hammer strikes;
classifying the segmented audio signals; and
providing instruction to a user regarding the fit of the instrument within the bone.
